(19) United States
(12) Patent Application Publication
(10) Pub. No.: US 2015/0265303 A1
(43) Pub. Date: Sep. 24, 2015

(54) SPINAL CORRECTION RELEASE SYSTEM AND METHOD

(71) Applicant: WARSAW ORTHOPEDIC, INC., WARSAW, IN (US)

(72) Inventors: Frank J. Schwab, New York, NY (US); Gary S. Lindemann, Collierville, TN (US)

(73) Assignee: WARSAW ORTHOPEDIC, INC., WARSAW, IN (US)

(21) Appl. No.: 14/221,037

(22) Filed: Mar. 20, 2014

(51) Int. Cl. A61B 17/32; A61B 17/3211; A61B 18/04

(52) U.S. Cl. CPC A61B 17/320016; A61B 18/04; A61B 17/3211; A61B 2017/320056; A61B 2017/320052; A61B 2018/00601

(57) ABSTRACT

A method for treating a spine comprises the steps of: creating a surgical pathway in tissue of a body along a posterior approach; delivering a cutting surface via the surgical pathway to adjacent a longitudinal element implanted with the body; and engaging the cutting surface with the longitudinal element. Systems and surgical instruments are disclosed.

### TECHNICAL FIELD

The present disclosure generally relates to medical devices for the treatment of musculoskeletal disorders, and more particularly to a surgical system and a method for correction of a spine disorder.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including abnormal growth or development of structures, trauma, disease and degenerative conditions caused by injury and aging. Spinal disorders can lead to deformity, cosmetic and functional impairment and symptoms including pain, nerve damage, and partial or complete loss of mobility.

Non-surgical treatments, such as medication, rehabilitation and exercise can be effective, however, may fail to address deformity or relieve the symptoms associated with these disorders. Surgical treatment of these spinal disorders includes correction fusion, fixation, discectomy, laminectomy and implantable prosthetics. Correction treatments used for positioning and alignment may employ implants such as rods, tethers and bone screws for stabilization of a treated section of a spine. This disclosure describes an improvement over these prior art technologies.

### SUMMARY

In one embodiment, a method for treating a spine is provided. The method comprises the steps of creating a surgical pathway in tissue of a body along a posterior approach; delivering a cutting surface via the surgical pathway to adjacent a longitudinal element implanted within the body; and engaging the cutting surface with the longitudinal element. In some embodiments, surgical instruments and systems are disclosed.

### BRIEF DESCRIPTION OF THE DRAWINGS

The present disclosure will become more readily apparent from the specific description accompanied by the following drawings, in which:

FIG. 1 is an axial view of components of one embodiment of a system in accordance with the principles of the present disclosure disposed with vertebrae;

FIG. 2 is a perspective view of a component of the system shown in FIG. 1;

FIG. 3 is a perspective view of components of one embodiment of a system in accordance with the principles of the present disclosure;

FIG. 4 is a perspective view of components of one embodiment of a system in accordance with the principles of the present disclosure;

FIG. 5 is a side view of components of one embodiment of a system in accordance with the principles of the present disclosure;

FIG. 6 is a side view of the components shown in detail A in FIG. 5;

FIG. 7 is a side view of the components shown in detail A in FIG. 5; and

FIG. 8 is a side view of the components shown in detail A in FIG. 5.

### DETAILED DESCRIPTION

The exemplary embodiments of the system and related methods of use disclosed are discussed in terms of medical devices for the treatment of musculoskeletal disorders and more particularly, in terms of a surgical system and method for treatment of a spine disorder. In one embodiment, the present disclosure provides a surgical system and method that release tension and/or excessive or over correction of an implanted correction system, which is employed to treat a spine disorder, as described herein. This configuration can avoid non-desirable surgery, such as, for example, a thoracotomy and/or tissue disruption or damage.

In some embodiments, the system and method employ a substantially posterior surgical approach and/or variation thereof and creates a substantially posterior surgical pathway to dispose a surgical instrument to engage components of an implanted correction system to release tension and/or avoid excessive or over correction. In some embodiments, the system releases tension in the components after a substantially desired correction and/or alignment has been achieved and/or tension becomes too great. For example, in cases, such as, a spinal tethering system that has been implanted with a child for several months or years and the maximum possible straightening has been applied to a scoliotic spine.

In one embodiment, a medical practitioner approaches an anterior tethering system from a posterior surgical approach. In one embodiment, the tethering system is cut to release tension in a selected tether. In some embodiments, the system is employed with a percutaneous surgical procedure, which may include a dilator. In one embodiment, the system is employed with a procedure that penetrates soft tissue and avoids a transverse process adjacent a selected region of a tether to be cut. In some embodiments, the system is employed with image guidance, such as, for example, fluoroscopy or CT scanning. See also, the examples and disclosure of systems, markers, implants and methods shown and described in U.S. patent application Ser. No. ______ (Attorney Docket No. C00006841.USU1) filed March ______, 2014, and published as U.S. patent application Publication Ser. No. ______, on ______, the entire contents of which being incorporated herein by reference.

In one embodiment, the system includes a dilator that is inserted adjacent a surgical site such that a cutting instrument can be disposed adjacent the selected region of the tether. In some embodiments, the dilator creates a posterior surgical pathway. In one embodiment, the practitioner positions a cutting tool through the surgical pathway such that a cutting surface is disposed adjacent to a spinal tethering system. In one embodiment, the cutting tool extends out of a cannula during a percutaneous procedure and the cutting tool cuts the tether to release tension in the tether. In one embodiment, the cutting tool is positioned around or through a transverse process. In some embodiments, the system comprises a cutting surface that includes a metallic wire that is heated. In some embodiments, the wire is inserted through a cannula to reach the location of the tether. In some embodiments, the cutting surface can extend out of the cannula and cut the tether. In some embodiments, the cutting surface captures the tether with the cannula such that the cannula is a fixed surface and the cutting surface cuts the tether with an edge of the cannula.

In some embodiments, the cutting surface comprises a guillotine configuration such that the cutting surface extends out of the cannula to grasp the tether and is pulled back in the cannula. When the tether is pulled back into engagement with the cannula, the cutting surface can dip the tether at an edge of the cannula where the cutting surface re-enters the cannula. In some embodiments, the cutting surface comprises a hook blade.

In one embodiment, the cutting surface comprises a surgical instrument having a ronguer configuration. In one embodiment, the cutting surface comprises a ronguer having a 90 degree angled flange, as described herein. In one embodiment, the ronguer defines a passageway configured for relative movement of the cutting surface. In some embodiments, the cutting surface may include an RF electro cautery device disposed with the passageway that engages the tether to sever the tether while minimizing the impact on adjacent tissue. In some embodiments, the cutting surface may include a heated knife disposed with the passageway that engages the tether to sever the tether while minimizing the impact on adjacent tissue. In some embodiments, the cutting surface includes an ultrasonic cutter, such as, for example, a harmonic scalpel. In some embodiments, the cutting surface may include a sharpened edge disposed with the passageway that engages the tether to sever the tether while minimizing the impact on adjacent tissue. In one embodiment, the ronguer includes a tip having a cavity configured to encapsulate a tether. In one embodiment, the ronguer includes a lower arm having a bullet nose shape. In one embodiment, the ronguer includes a tip that captures the tether. It is envisioned that the tip prevents engagement of the cutting surface with tissue.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context dearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, vessels, ligaments, tendons, cartilage and/or bone unless specifically referred to otherwise.

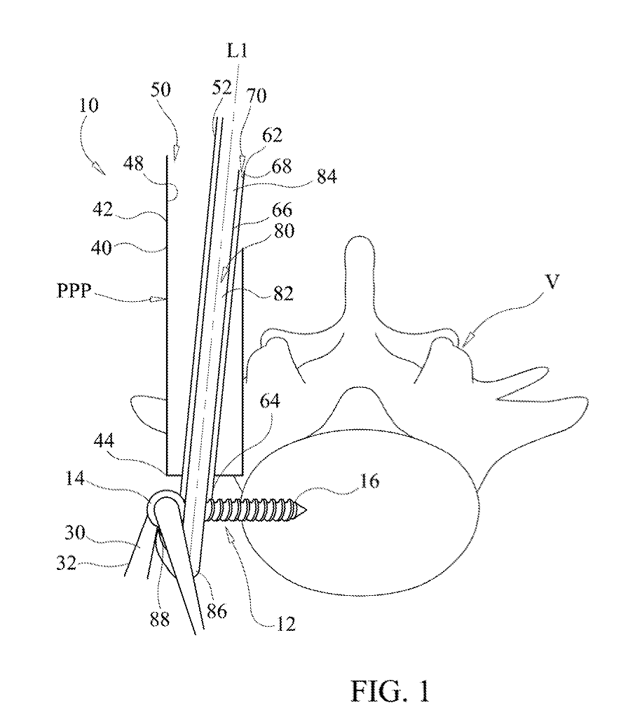

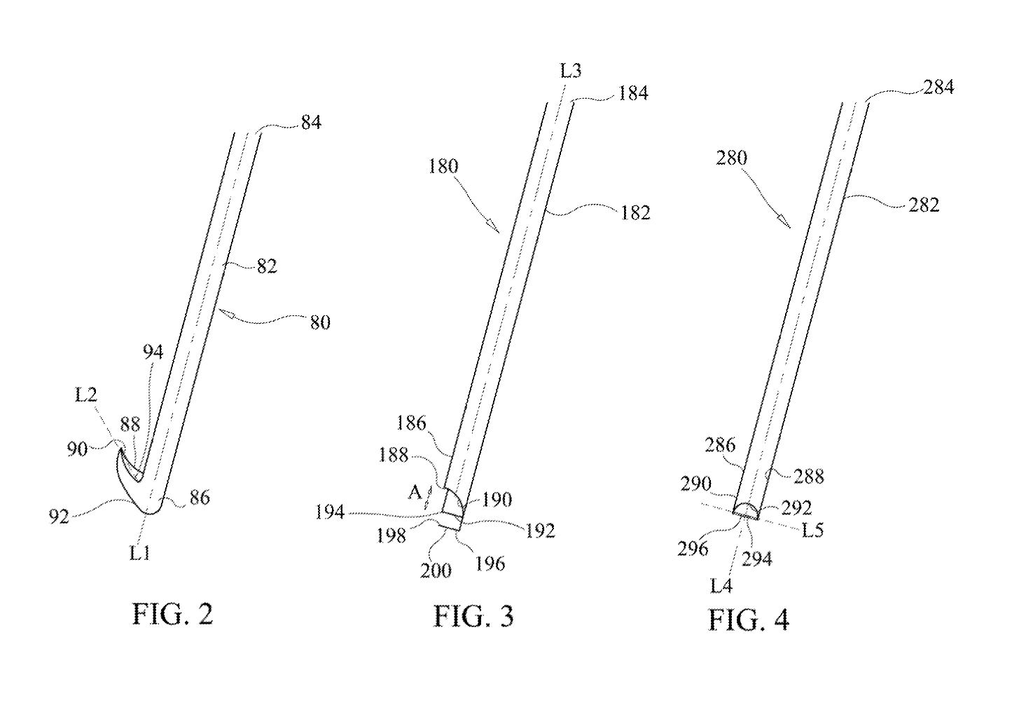

The following discussion includes a description of a surgical system including surgical instruments, related components and methods of employing the system in accordance with the principles of the present disclosure. Alternate embodiments are also disclosed. Reference is made in detail to the exemplary embodiments of the present disclosure, which are illustrated in the accompanying figures. Turning to FIGS. 1-2, there are illustrated components of a method and system, such as, for example, a surgical correction system 10 in accordance with the principles of the present disclosure.

A spinal construct including, such as for example, a tissue fastener, such as, for example, a bone screw 12 is fixed with vertebrae V. In some embodiments, system 10, which may include the spinal construct, and/or the spinal construct may include one or a plurality of tissue fasteners. In some embodiments, bone screw 12 may be engaged with tissue in various orientations, such as, for example, series, parallel, offset, staggered and/or alternate vertebral levels. In some embodiments, one or more tissue fasteners may comprise multi-axial screws, sagittal angulation screws, pedicle screws, mono-axial screws, uni-planar screws, facet screws, fixed screws, tissue penetrating screws, conventional screws, expanding screws, wedges, anchors, buttons, dips, snaps, friction fittings, compressive fittings, expanding rivets, staples, nails, adhesives, posts, fixation plates and/or posts.

Bone screw 12 comprises a first portion, such as, for example, a head 14 and a second portion, such as, for example, an elongated shaft 16 configured for penetrating tissue. Head 14 includes a receiving portion configured for disposal of an implant, such as, for example, a tether 30. The spinal construct includes a longitudinal element, such as, for example, tether 30. Tether 30 is attached with and extends along an anterior portion of vertebrae V.

The spinal construct forms one or more components of a correction treatment and/or correction system used for positioning and alignment for stabilization of a treated section of vertebrae V, which is implanted with vertebrae V in a prior surgical procedure. In some embodiments, system 10 may be employed with a surgical procedure for implanting components of the correction system. In one embodiment, tether 30 is connected with heads 14 of bone screws 12 causing a tension in tether 30 and/or vertebrae V. In some embodiments, the spinal construct, for example, tether 30 and/or a tension thereof is employed to displace, pull, twist or align vertebrae V as part of a correction system and treatment. In some embodiments, the spinal construct, for example, tether 30 may have previously had a tension with vertebrae V and no longer has a tension for engagement with the components of system 10. In some embodiments, the spinal construct, for example, tether 30 is implanted in a prior surgical procedure to provide stabilization and/or correction to vertebrae V and the components of system 10 are engageable with tether 30, as described herein, to release stabilization and/or correction forces presently and/or previously applied to vertebrae V.

In some embodiments, tether 30 has a flexible configuration, which includes movement in a lateral or side to side direction and prevents expanding and/or extension in an axial direction upon fixation with vertebrae. In some embodiments, all or only a portion of tether 30 may have a semi-rigid, rigid, flexible or elastic configuration, and/or have elastic and/or flexible properties such as the elastic and/or flexible properties corresponding to the material examples described above such that tether 30 provides a selective amount of expansion and/or extension in an axial direction. Tether 30 can include a plurality of separately attachable or connectable portions or sections, such as bands or loops, or may be monolithically formed as a single continuous element.

Tether 30 has an outer surface 32 and a uniform thickness/diameter. In some embodiments, outer surface 32 may have various surface configurations, such as, for example, rough, threaded for connection with surgical instruments, arcuate, undulating, porous, semi-porous, dimpled, polished and/or textured. In some embodiments, the thickness defined by tether 30 may be uniformly increasing or decreasing, or have alternate diameter dimensions along its length. In some embodiments, tether 30 may have various cross section configurations, such as, for example, oval, oblong, triangular, rectangular, square, polygonal, irregular, uniform, non-uniform, variable and/or tapered.

In some embodiments, tether 30 may have various lengths. In some embodiments, tether 30 may be braided, such as a rope, or include a plurality elongated elements to provide a predetermined force resistance. In some embodiments, tether 30 may be made from autograft and/or allograft and be configured for resorbable or degradable applications. In some embodiments, the longitudinal element can include a spinal rod.

System 10 includes a surgical instrument, such as, for example, a dilator 40. Dilator 40 is configured to dilate fascia and muscle fiber around the surgical site to create a posterior surgical pathway, such as, for example, a posterior percutaneous passageway PPP extending between a surface of the patient body to adjacent tether 30. In one embodiment, the tissue includes soft tissue adjacent a transverse process. In one embodiment, the tissue includes bony tissue adjacent the transverse process.

Dilator 40 extends between a proximal end 42 and a distal end 44. In some embodiments, end 44 includes a tip configured to punch through fascia so fascia could then be expanded. Dilator 40 includes an inner surface 48 defining a passageway 50 configured to receive a surgical instrument or tool, such as, for example, those described herein.

System 10 includes a surgical instrument, such a cannula 52 having a circumferential cross section. Cannula 52 extends between an end 62 and an end 64. In one embodiment, end 62 may include a gripping surface 66 that may be such as, for example, rough, arcuate, undulating, mesh, porous, semi-porous, dimpled and/or textured. Cannula 52 includes an inner surface 68 that defines a cavity 70 configured for disposal of the cutting instrument, as discussed herein. In one embodiment, dilator 40 includes cannula 52.

System 10 includes a surgical instrument, such as, for example a cutting instrument 80. Cutting instrument 80 includes an elongated shaft 82 extending between an end 84 and an end 86 and defines a longitudinal axis L1. In one embodiment, end 84 may include a gripping surface that may be, such as, for example, rough, arcuate, undulating, mesh, porous, semi-porous, dimpled and/or textured to facilitate manipulation of cutting instrument 80. End 86 includes a cutting surface 88 that is hooked shaped. In some embodiments, cutting surface 88 may have various configurations such as, for example, round, oval, oblong, triangular, irregular, uniform or non-uniform.

Cutting surface 88 includes an inner surface 90 and an outer surface 92. Surface 90 includes a cutting element, such as, for example, a blade 94 configured to cut and/or sever a longitudinal element, such as, for example, tether 30. Surface 90 defines an axis L2 disposed at an angle transverse to axis L1 to provide leverage for cutting tether 30 upon manipulation of end 84. In some embodiments, blade 94 may be disposed in various orientations relative to axis L2, such as, for example, side-by-side, parallel, perpendicular or angular. Surface 92 is smooth and/or rounded such that surface 92 prevents engagement of the cutting surface with tissue adjacent vertebrae V. In one embodiment, cutting surface 88 can be extended and retracted into cannula 52 and/or into engagement with a surface of cannula 52 to cut and/or sever tether 30. In one embodiment, cutting surface 88 may be guided via imaging guidance, as described herein.

In assembly, operation and use, system 10, similar to the systems and methods described herein, is employed subsequent a surgical correction procedure employing a surgical correction system, similar to those described herein. In some embodiments, system 10 may be employed concurrently with the surgical correction procedure. For example, the surgical correction system may be employed in surgical procedures for treating disorders of the spine, such as, for example, a correction treatment to treat child/adolescent idiopathic scoliosis and/or Scheuermann's kyphosis of a spine. In some embodiments, one or al of the components of system 10 can be delivered as a pre-assembled device or can be assembled in situ.

The surgical correction treatment includes a spinal construct, similar to that described herein, that has been implanted with vertebrae V in a prior surgical procedure. The spinal construct is used for positioning and alignment for stabilization of a treated section of vertebrae V. Tether 30 is connected with heads 14 of bone screws 12 causing a tension in tether 30 and/or vertebrae V to displace, pull, twist or align vertebrae V as part of a correction system and treatment. In some embodiments, tether 30 is implanted in the prior surgical procedure to provide stabilization and/or correction to vertebrae V.

In use, to release tension and/or excessive or over correction of the implanted correction system, which includes tether 30 affixed with vertebrae V via bone screws 12, a medical practitioner obtains access to a surgical site including vertebrae V via a posterior surgical approach, as described herein. In some embodiments, the surgical site may be accessed in any appropriate manner, such as through incision and retraction of tissues. In some embodiments, system 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area.

An incision is made in the body of a patient and a surgical pathway, such as, for example, pathway PPP is created adjacent the spinal construct, as described herein, for employment with system 10. Dilator 40 is placed into the incision and inserted into pathway PPP from a posterior surface of the body of a patient and translated along tissue along a posterior approach such that distal end 44 is disposed adjacent a selected portion of the spinal construct, for example, a portion of tether 30. In some embodiments, dilator 30 is positioned via image guidance, such as, for example, computed tomography, fluoroscopy, magnetic resonance, positron emission tomography, ultrasound, or x-ray scans taken prior to or during the surgical procedure so as to minimize damage to tissue. For example, image guidance is provided to minimize damage to tissue surrounding a surgical site during placement of the components of system 10 and to aid in positioning dilator 40, cannula 52 and/or cutting instrument 80.

End 62 is manipulated and cannula 52 is disposed with passageway 50 and delivered through dilator 40 adjacent the selected portion of tether 30. End 64 is disposed to extend from dilator 40. Cutting instrument 80 is disposed with cavity 70 and delivered through cannula 52 adjacent the selected portion of tether 30. Cutting instrument 80 is positioned such that cutting surface 88 is disposed adjacent the portion of tether 30 to be cut. Manipulation of cutting instrument 80 causes blade 94 to cut tether 30 and release the tension in tether 30. In one embodiment, tether 30 is cut completely through its thickness. In one embodiment, tether 30 is cut only partially through its thickness. In some embodiments, cutting instrument 80 is engageable with tether 30, as described herein, to release stabilization and/or correction forces presently and/or previously applied to vertebrae V.

Upon completion of a procedure, as described herein, the surgical instruments, assemblies and non-implanted components of system 10 are removed and the incision(s) are closed. One or more of the components of system 10 can be made of radiolucent materials such as polymers. Radiopaque markers may be included for identification under x-ray, fluoroscopy, CT or other imaging techniques. In some embodiments, the use of surgical navigation, microsurgical and image guided technologies may be employed to access, view or repair spinal aspects, with the aid of system 10.

In some embodiments, the components of system 10 may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential.

In one embodiment, as shown in FIG. 3, system 10, similar to the systems and methods described above with regard to FIGS. 1 and 2, includes a cutting instrument 180, similar to cutting instrument 80 described herein. Cutting instrument 180 includes an elongated shaft 182 extending between an end 184 and an end 186 and defines a longitudinal axis L3. In one embodiment, end 184 may include a gripping surface that may be, such as, for example, rough, arcuate, undulating, mesh, porous, semi-porous, dimpled and/or textured to facilitate manipulation of cutting instrument 180. Shaft 182 includes an inner surface 188 that defines a cavity 190 configured for disposal of a cutting surface 192. Cutting surface 192 includes a cutting element, such as, for example, a blade 194 configured to cut and/or sever a longitudinal element, such as, for example, tether 30, described herein. Blade 194 extends along axis L3. In some embodiments, blade 194 may be disposed in various orientations relative to axis L3, such as, for example, side-by-side, parallel, transverse, perpendicular or angular. Blade 194 axially translates from end 186 along axis L3 in a guillotine motion along the path shown by arrows A to cut tether 30. Cutting surface 192 is disposed within shaft 182 and extends out to cut tether 30 and retracted into shaft 182. End 186 includes a flange 196. Flange 196 includes an inner surface 198 and an outer surface 200. Surface 198 is configured as a stop for cutting surface 192. Surface 200 prevents engagement of the cutting surface with tissue adjacent vertebrae V.

In one embodiment, as shown in FIG. 4, system 10, similar to the systems and methods described herein, includes a cutting instrument 280, similar to cutting instrument 80 described herein. Cutting instrument 280 includes an elongated shaft 282 extending between an end 284 and an end 286 and defines a longitudinal axis L4. Shaft 282 includes an inner surface 288 and an outer surface 290. Surface 288 defines a cavity 292 configured for disposal of a cutting surface 294. Cutting surface 294 is disposed in cavity 292 and defines an axis L5 transverse to axis L4. Cutting surface 294 includes a heated metallic wire 296 configured to cut tether 30 to release the tension in tether 30.

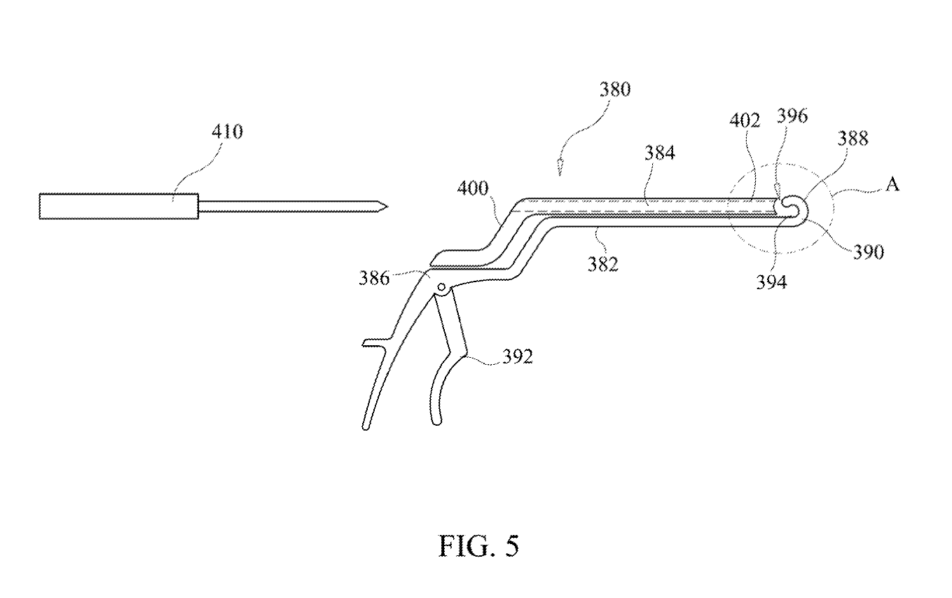

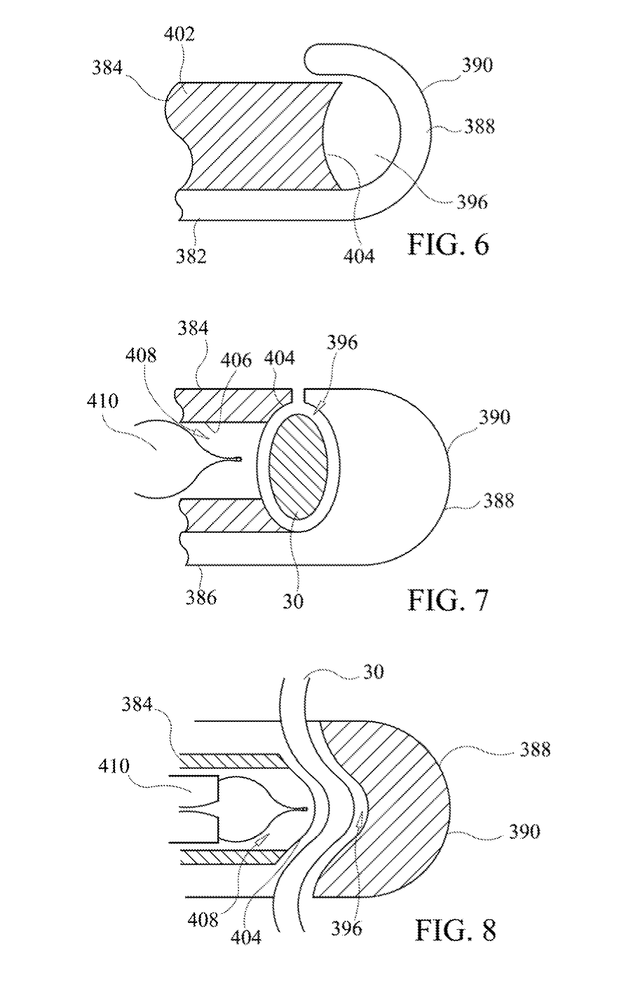

In one embodiment, as shown in FIGS. 5-8, system 10, similar to the systems and methods described herein, includes a cutting instrument 380, similar to the cutting instruments described herein. Cutting instrument 380 includes an elongated shaft 382 and a sleeve 384, as shown in FIG. 5. Shaft 382 extends between a proximal end 386 and a distal end 388. Shaft 382 includes blunt tip 390 positioned at end 388 configured to prevent non-desirable engagement of a cutting surface with tissue, as described herein. Shaft 382 includes an inner surface 394 that defines a cavity 396, as shown in FIG. 6. Cavity 396 is configured to capture tether 30, as shown in FIG. 7. End 386 includes an actuator, such as, for example, a lever 392 configured to translate sleeve 384 relative to shaft 382 to capture tether 30 within cavity 396.

Sleeve 384 extends between proximal end 400 and distal end 402. Sleeve 384 is configured for translation along shaft 382 to capture tether 30. In one embodiment, sleeve 384 surrounds shaft 382. In one embodiment sleeve 384 is releasably engaged with a portion of shaft 382. End 402 includes a surface 404 configured to capture tether 30 between end 402 and end 388. Sleeve 384 is configured to translate along a portion of shaft 382 such that sleeve 384 moves towards end 388 of shaft 382 to capture tether 30 between surface 404 and end 388. In one embodiment, surface 404 is arcuate such that it has a contoured fit around tether 30. In one embodiment, surface 404 may be, such as, for example, rough, undulating, mesh, porous, semi-porous, dimpled and/or textured to facilitate capture of tether 30.

Sleeve 384 includes an inner surface 406 that defines a passageway 408. Passageway 408 is configured for disposal and relative translation of a cutting instrument, such as, for example, a heated wire 410, as shown in FIGS. 7 and 8. Wire 410 is manipulated for translation to extend through passageway 408 such that wire 410 engages and cuts and/or severs tether 30 to release tension in tether 30, as described herein. Wire 410 includes electrical leads, as shown in FIG. 8, connected to an electrical power source such that wire 410 generates heat and is applied to tether 30 to sever tether 30. In some embodiments, the cutting instrument may comprise an RF electro cautery device having a cutting surface for cutting and/or severing a longitudinal element. In some embodiments, the cutting instrument may comprise an ultrasonic cutter having a cutting surface, such as, for example, a harmonic scalpel for cutting and/or severing a longitudinal element.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for treating a spine, the method comprising the steps of:
creating a surgical pathway in tissue of a body along a posterior approach;
delivering a cutting surface via the surgical pathway to adjacent a longitudinal element implanted within the body; and
engaging the cutting surface with the longitudinal element.

2. A method as recited in claim 1, wherein the step of creating includes providing a dilator to space the tissue to create the surgical pathway.

3. A method as recited in claim 2, wherein the dilator includes a cannula having a circumferential cross-section and an inner surface defining a cavity configured for disposal of the cutting surface.

4. A method as recited in claim 1, further comprising the step of providing image guidance to facilitate positioning of the cutting surface.

5. A method as recited in claim 1, wherein the tissue includes soft tissue adjacent a transverse process.

6. A method as recited in claim 1, wherein the tissue includes bony tissue adjacent a transverse process.

7. A method as recited in claim 1, wherein the cutting surface includes a heated metallic wire.

8. A method as recited in claim 1, wherein the cutting surface includes a guided blade.

9. A method as recited in claim 1, wherein the cutting surface includes a harmonic scalpel.

10. A method as recited in claim 1, wherein the cutting surface includes a hook-shaped blade.

11. A method as recited in claim 1, wherein the longitudinal element includes a tether.

12. A method as recited in claim 1, wherein the longitudinal element includes a tether, the tether being implanted with an anterior portion of vertebrae of the body.

13. A method as recited in claim 1, wherein the surgical pathway comprises a percutaneous passageway extending between a surface of the body to adjacent the longitudinal element.

14. A method for treating a spine, the method comprising the steps of:
providing a dilator to space tissue of a body and create a percutaneous passageway in the tissue along a posterior approach;
delivering a cutting surface along the passageway to adjacent a tether implanted within the body, the tether having a tension; and
engaging the cutting surface with the tether to release the tension.

15. A method as recited in claim 14, wherein the cutting surface includes a metallic wire.

16. A method as recited in claim 14, wherein the cutting surface includes a guided blade.

17. A method as recited in claim 14, wherein the cutting surface includes a hook-shaped blade.

18. A surgical system for treating a spine, the system comprising:
a posterior approach dilator including an inner surface defining a pathway and an end surface disposable adjacent a longitudinal element implanted within a body; and
a cutting surface movable along the pathway and into an orientation relative to the dilation for engaging the longitudinal element.

19. A surgical system as recited in claim 18, wherein the cutting surface includes a metallic wire.

20. A surgical system as recited in claim 18, wherein the cutting surface includes a guided blade.
